(19) United States
(12) Patent Application Publication
(10) Pub. No.: US 2020/0154985 A1
(43) Pub. Date: May 21, 2020

(54) ENDOSCOPIC PROBE HAVING ROTATING CORE WITH BUILT-IN ENCODER

(71) Applicant: Canon U.S.A. Inc., Melville, NY (US)

(72) Inventor: Mitsuhiro Ikuta, Cambridge, MA (US)

(21) Appl. No.: 16/192,398

(22) Filed: Nov. 15, 2018

(51) Int. Cl. A61B 1/00; A61B 5/00; G02B 23/24; A61B 1/06; A61B 1/07

(52) U.S. Cl. CPC A61B 1/00172; A61B 1/00096; A61B 1/00167; A61B 1/00174; A61B 1/00126; G02B 23/2423; A61B 1/0676; A61B 1/07; A61B 1/0623; A61B 5/0062

(57) ABSTRACT

An endoscopic probe includes a rotatable core for guiding first light from a first light source to a sample; a non-rotatable sheath arranged concentrically around the rotatable core; and a plurality of fibers surrounding the distal end of the non-rotatable sheath. The rotatable core includes a plurality of patterns built-in at the distal end thereof. Some of the fibers are used to detect the first light scattered by the sample, some fibers are used to irradiate the patterns with second light, and some fibers are used to collect the second light reflected from the patterns. The fibers are aligned with plurality of patterns to collect the reflected second light as the rotatable core rotates, and the rotation position of the rotatable core is determined by correlating intensity variation the reflected light with time collection. NURD of a SEE image is compensated based on the position information.

### CROSS-REFERENCE TO RELATED APPLICATIONS

n/a

### BACKGROUND INFORMATION

### Field of the Disclosure

The present disclosure relates to medical imaging devices. More particularly, the disclosure exemplifies endoscopic probes with a built-in optical encoder to measure non-uniform rotational distortion (NURD). Methods and systems for measuring and compensating NURD are also disclosed.

### Description of Related Art

Imaging modalities that use a mechanically rotated optical probe (e.g., endoscope or catheter) to scan a bodily lumen (e.g., an artery) often suffer from image degradation due to non-uniform rotational distortion (NURD). In this regard, successful optical scanning of internal structures of a subject requires overcoming several challenges including motion artifacts associated with patient and/or organ movement, and performing accurate NURD measurement. Several techniques have been proposed to measure and correct NRUD, but most conventional techniques fall short in accurately measuring the cause of the distortion. A known method measures the rotational speed of a catheter by determining the statistical variation in the speckle between adjacent A-lines. Other methods use cross correlation or phase information of successive frames but generally require highly correlated A-line data. These methods are data intensive and time consuming; see, e.g., U.S. Pat. Nos. 9,668,638 and 9,237,851, and pre-grant publication US 2013/0107274. In addition, U.S. Pat. No. 8,712,506 discloses an optical encoder integrated into devices with elongate, flexible rotary shafts, and the use of the output of the rotary encoder to improve co-registration of multi-modality images.

More recently, the present applicant has disclosed other methods for addressing NURD. For example, pre-grant publication US 2017/0360398 discloses exemplary probes including longitudinal marker elements arranged parallel to the probe axis for measuring NURD and ring marker elements arranged concentric to the probe axis for measuring non-uniform linear distortion (NULD). In this publication, light for NURD detection comes from rotating parts which are the same rotating optics for side-view imaging. In addition, pre-grant publication US 2018/0064396 proposes arranging electrodes concentrically to rotating and non-rotating elements of an imaging probe. The electrodes are rotationally aligned and form a capacitive sensor that can sense the rotation angle of the inner core compared to a cylindrical tube surrounding the core.

However, these known designs and techniques have significant drawbacks in particular, with respect to spectrally encoded endoscopy (SEE) probes designed for forward viewing applications. Forward viewing (or front viewing) SEE probes are particularly advantageous for endoscopic imaging applications such as, ear, nose and throat (ENT) examinations, laparoscopy, orthopedic endoscopic surgery (arthroscopy), and pediatric surgery. However, in a forward-viewing SEE probe, illumination light is projected to the front of the probe such that at least one color wavelength or at least one diffracted order of light propagates substantially parallel to the probe axis. Therefore, in the case of a forward viewing SEE probe, it is difficult to use the same light for illuminating the sample and for detecting the rotation of the probe because the illumination light needs to be projected forward while rotation should be measured radially. In addition, due to the need to miniaturize the size and lower the cost of a SEE probe, the use of capacitive sensors becomes less desirable as it can increase the size and weight of the probe. Furthermore, other methods that use cross correlation or phase information of successive image frames are data intensive and time consuming. Moreover, in ultraminiature SEE endoscopes, space for an encoder is limited. In addition to the above, in ultraminiature SEE endoscopes, the longer a rotational drive shaft, or the smaller the diameter the drive shaft, the more likely that NURD will occur.

Accordingly, it can be beneficial to address and/or overcome at least some of the deficiencies indicated herein above, and thus provide a new SEE probe having forward direction view, and an apparatus to use such a probe with appropriately corrected non-uniform rotational distortion.

### SUMMARY OF EXEMPLARY EMBODIMENTS

According to at least one embodiment of the present disclosure, there is provided an endoscopic optical probe, comprising: a rotatable core for guiding first illumination light from a first light source towards an imaging target, the rotatable core extending from a proximal end to a distal end along an axis and having a plurality of patterns built-in at the distal end thereof; a non-rotatable sheath arranged concentrically around the rotatable core and also extending from the proximal end to the distal end; and one or more optical waveguides disposed on the outer surface of the non-rotatable sheath at the distal end thereof, wherein the one or more waveguides are configured to guide second illumination light from a second light source onto the plurality of patterns and collect light reflected from the plurality of patterns, wherein the plurality of patterns are rotationally aligned with the waveguides such that the waveguides can collect the reflected light as a function of a rotation position of at least first and second patterns on the rotatable core, and wherein the rotation position is determined by intensity variation the reflected light.

According to one embodiment, the endoscopic optical probe is a forward viewing spectrally encoded endoscopy (SEE) probe. The probe includes a rotatable core for guiding first light from a first light source to an imaging target; a non-rotatable sheath arranged concentrically around the rotatable core; and a plurality of fibers surrounding the distal end of the non-rotatable sheath. The rotatable core includes a plurality of patterns built-in at the distal end thereof. Some of the fibers are used as detecting fibers to detect the first light scattered by the imaging target, and some fibers are used as waveguides to irradiate and collect second light from the plurality of patterns. SEE illumination optics is arranged inside the rotatable core to irradiate the imaging target. The rotatable core has at least first and second patterns different from each other; the patterns are different in reflectivity or other characteristic. When the second light irradiates the first and second patterns at different times during rotation, rotation position of the rotatable core is determined by intensity variation of the detected second light. A processor can be configured (programmed) with compensating algorithms to compensate NURD in a SEE image.

These and other objects, features, and advantages of the present disclosure will become apparent upon reading the following detailed description of exemplary embodiments of the present disclosure, when taken in conjunction with the appended drawings, and provided claims.

### BRIEF DESCRIPTION OF DRAWINGS

Further objects, features and advantages of the present disclosure will become apparent from the following detailed description when taken in conjunction with the accompanying figures showing illustrative embodiments of the present disclosure.

FIG. 1A shows an exemplary embodiment of a SEE probe system 100 according to the present disclosure. FIG. 1B shows a functional diagram of the image processor 230 applicable to the SEE probe system 100.

FIG. 2 illustrates a portion of the SEE probe too showing a schematic of a rotating portion of a probe 120 as it might be used in the embodiment illustrated in FIG. 1.

FIG. 3 illustrates the distal end of the probe 120 showing a built-in optical encoder as it might be used in the embodiment illustrated in FIG. 1.

FIGS. 4A, 4B, 4C and 4D show axial views of a portion of the SEE probe 100 illustrating exemplary arrangements of a built-in optical encoder as it might be seen near the distal end of probe 120 the direction of the probe axis Ox.

FIG. 5A shows exemplary intensity modulation rotation signals detected at the second detector 250 as a function of time. FIG. 5B shows exemplary image data obtained by spectrometer 220 as a function of wavelength (spectrum). FIG. 5C shows exemplary compensated image data in angular coordinates.

FIG. 6 shows an exemplary block diagram of image processing, and NURD measurement and compensation executed by the image processor 230 of the SEE probe system 100.

Throughout the figures, the same reference numerals and characters, unless otherwise stated, are used to denote like features, elements, components or portions of the illustrated embodiments. Moreover, while the subject disclosure will now be described in detail with reference to the figures, it is done so in connection with the illustrative exemplary embodiments. It is intended that changes and modifications can be made to the described exemplary embodiments without departing from the true scope and spirit of the subject disclosure as defined by the appended claims.

### DETAILED DESCRIPTION OF EXEMPLARY EMBODIMENTS

The embodiments disclosed herein are based on an object of addressing one or more of the issues discussed in the Background section. According to the disclosed embodiments, it is advantageous to provide an endoscopic probe having a built-in encoder, and an apparatus to use such a probe with appropriate correction of non-uniform rotational distortion. Exemplary embodiments are described as referring to spectrally encoded endoscopy (SEE). However, the novel arrangements of a built-in encoder and the novel image processing algorithms disclosed herein are not limited to SEE probes. The embodiments disclosed herein can also be applicable to various imaging modalities that use a mechanically rotated optical probe (e.g., endoscope or catheter) to scan a bodily lumen (e.g., an artery) using a fiber optic rotary joint (FORJ) or similar rotary devices.

### <SEE Imaging Probe>

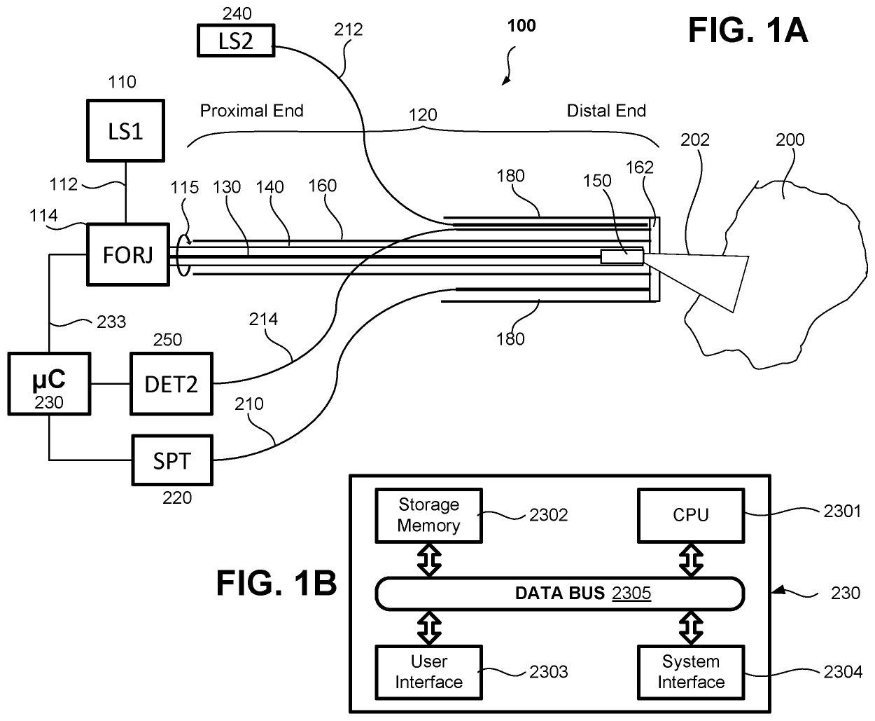

FIG. 1A shows an exemplary embodiment of a SEE probe system 100 according to the present disclosure. This exemplary SEE probe system 100 includes a first light source 110 (LS1) which is a broadband light source, an endoscope or catheter which is generally referred to as a probe 120, a spectrometer 220 (SPT) or first detector capable of measuring spectral components of broadband light emitted by the light source 110, and a micro-computer (μC) or image processor 230. FIG. 1B shows a functional diagram of the image processor 230. The probe system 100 also includes a second light source 240 (LS2) and a second detector 250 (DET2).

The spectrometer 220 may include (or can be replaced by) a first detector capable of measuring spectral components of broadband light 116 emitted by the light source 110. The broadband light has sufficient bandwidth to allow for spatial resolution along a spectrally dispersed dimension. In some embodiments, the broadband light 116 emitted by light source 110 is a broadband visible light in the rage of about 400-800 nm which includes a blue band of light (including wavelengths λ_{B1} to λ_{BN}), a green band of light (including wavelengths λ_{G1} to λ_{GN}), and a red band of light (including wavelengths λ_{R1} to λ_{RN}). For example, the blue band includes light in a wavelength range of 400-500 nm, the green band includes light in a wavelength range of 500-600 nm, and the red band includes light in a wavelength of 600-800 nm. In other embodiments, the wavelengths of the broadband light are optimized for identifying specific features such as blood, tissue, etc., and may extend into the near infra-red (near IR) region.

The broadband light source 110 may include a plurality of light sources or may be a single light source. The broadband light source 110 may include one or more of a laser, an OLED, a LED, a halogen lamp, an incandescent lamp, a supercontinuum light source pumped by a laser, and/or a fluorescent lamp. The broadband light source 110 may be any light source that provides light which can then be split up into at least three bands in which each band is further dispersed to provide light which is then used for spectral encoding of spatial information. The broadband light source 110 may be coupled by a light guiding component or may be free-space coupled to another component of the SEE probe system 100.

The light guiding component may be a source optical fiber 112 or some other optical waveguide which serves to connected the light source 110 to the probe 120. The source optical fiber 112 may be a single-mode fiber, double clad fiber, a multi-mode fiber. The source optical fiber 112 may include one or more fibers. Preferably, a single fiber is used as the source optical fiber 112. The source optical fiber 112 is connected to an illumination fiber 130 of the optical probe through a fiber optic rotary junction (FORJ) 114. The illumination fiber 130 is arranged inside a drivecable 140. The drivecable 140 delivers torque from its proximal end to distal end. The drivecable 140 can be flexible or rigid.

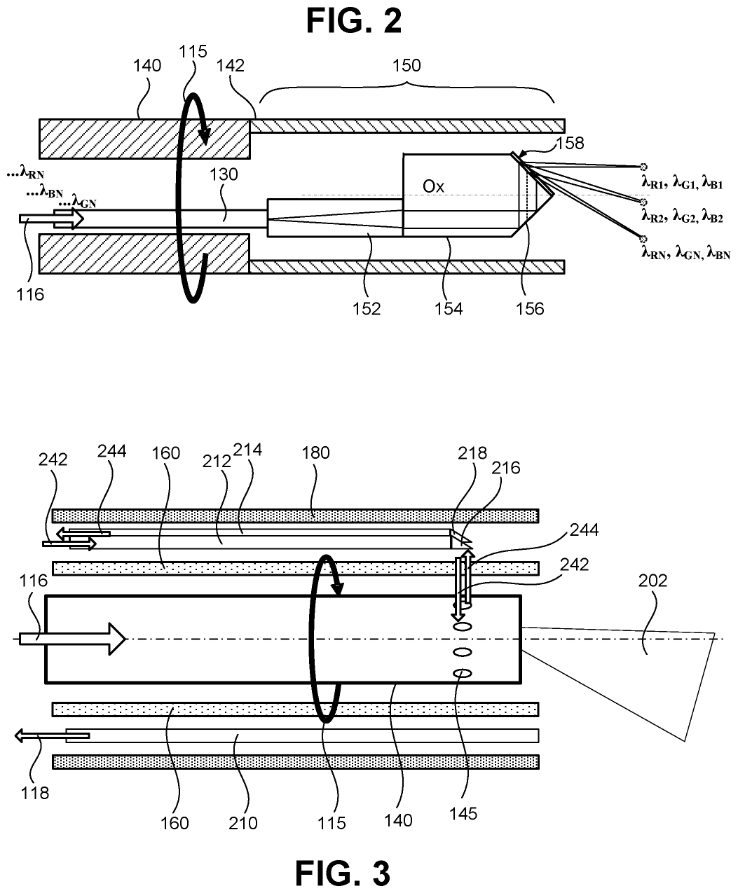

FIG. 2 is an illustration of a portion of the SEE probe 100 showing a schematic of a rotating portion of the probe 120 as it might be used in the embodiment illustrated in FIG. 1. The rotating portion (rotatable core) of the probe 120 includes illumination optics comprising an illumination fiber 130 (light guiding component), a lens 152 (light focusing component), and a grating 158 (diffractive component) arranged in this order along an axis Ox and enclosed in the drivecable 140 and can 142. Probe distal optics 150 are attached at the tip or endface of the illumination fiber 130. The illumination fiber 130 transmits broadband light 116 to the distal optics 150. The drivecable 140 may include a structured cylindrical protector (can) 142 at the distal tip to contain inside thereof the probe distal optics 150. Parts of the probe 120, for example the illumination fiber 130, the drivecable 140, and the probe distal optics 150, can be rotated or oscillated as by the rotary junction 114 in the direction indicated by the arrow 115.

The drivecable 140 is inside of an inner sheath 160. The inner sheath 160 does not rotate while the drivecable 140 is rotated. Therefore, a certain tolerance is necessary between an inner diameter of the sheath 160 and an outer diameter of the drive cable 140. In addition, the inner surface of the inner sheath 160 is preferably smooth to reduce friction between the drivecable 140 and the inner sheath 160. For example, the inner surface of the inner sheath 160 can be coated with Teflon® or other similar material. In general, at least one of the inner surface of the inner sheath 160 or the outer surface of the drivecable 140 is coated with a buffer solution and/or hydrophilic coating.

As shown in FIG. 2, the probe distal optics 150 includes a light focusing component 152 and a transparent spacer 154. The end face of illumination fiber 130 can be attached or fused to the light focusing component 152. The illumination fiber 130 can be centered or off-centered with the optical axis of light focusing component 152. In the former case, the light direction of at least part of the illumination beam is substantially parallel to optical axis (Ox) of the probe 120. In the latter case, the light direction of at least part of the illumination beam is angled with respect to the optical axis depending on the off-axis amount. In this embodiment of FIG. 2, broadband light having components in the red, green, and blue (R, G, B) spectrum is coupled from the light source 110 into the illumination fiber 130 via the FORJ 114.

The light focusing component 152 may be, for example, a GRIN lens or a ball lens. The spacer 154 may be made of, but is not limited to, glass, heat-curable resin, UV-curable resin, or plastic. The spacer 154 can have a reflective surface or mirror 156 and a diffractive component or grating 158. The mirror 156 may be achieved, for example, by applying a reflective coating (e.g., metal coating), or by using total internal reflection on a part of spacer 154. The grating 158 may be fabricated by techniques such as dry-etching, wet-etching, nano-imprint, and soft lithography. The grating 158 may be formed directly or may be stamped on the spacer 154. For example, the spacer 154 with grating 158 can be fabricated by dicing and angle-polishing etched glass grating. The grating 158 may be, but is not limited to, a binary grating, a blazed grating, or a holographic grating.

The blue band of light (λ_{B1} to λ_{BN}), the green band of light (λ_{G1} to λ_{GN}), and red band of light (λ_{R1} to λ_{RN}) are incident on the grating 158 at substantially the same incident angle θᵢ.

The diffraction angle θ_{d} may be determined by a known grating equation such as Equation (1):

nᵢ sin θᵢ+n_{d} sin θ_{d}=−mGλ (1)

where, nᵢ is the refractive index of the material on the incident side of the grating 158; n_{d} is the refractive index of the material on the diffraction side of the grating; m is the diffraction order; G is spatial frequency (slits or lines per unit length) of the grating 158, and λ is wavelength of the light.

In an exemplary embodiment where the material on the diffraction side of the grating is assumed to be air (n_{d}=1), the diffraction conditions of grating 158 may be designed according to the following parameters: nᵢ=1.5037; n_{d}=1; θᵢ=42.810; and G=860/mm. In another exemplary embodiment, the diffraction conditions may be based on the following parameters: nᵢ=1.4713; n_{d}=1; θᵢ=42.7°; and G=650/mm. In some embodiments, the grating 158 is designed so that the absolute value of the product of the spatial frequency G and the diffraction order for green light m_{G}, |G*m_{G}|, is more than 2000/mm, 2500/mm, or 3000/mm.

It is understood that, while the red, green, and blue light bands are incident on the grating 158 at substantially the same incident angle θᵢ, there may be some differences due to dispersion and/or manufacturing tolerances. Wavelength shift on target due to those differences can be compensated in the image reconstruction process, if necessary. For example, the incidence angle θᵢ of the red light and the blue light may vary by about 1.0° or less. As used herein “substantially the same incident angle” in the context of the incident angle θᵢ means a variation of 1.0° or less than 1.0°.

In order to make the blue band of illumination light, green band of illumination light, and red band of illumination light overlap on the sample 200 to form a line image, the diffraction order for the blue band of light m_{B}, the diffraction order for the green band of light m_{G}, and the diffraction order for the red band of light m_{R} are restricted to satisfy Equations (2) below:

|m_{B}|=|m_{G}|+1

|m_{R}|=|m_{G}|−1

{m_{R},m_{G},m_{B}∈ [symbol] |sgn(m_{R})=sgn(m_{G})=sgn(m_{B}),(m_{R},m_{G},m_{B})≠0} (2)

In Equation (2), [symbol] is an integer, and expression “sgn(x)” denotes a sing of “x”, where the sign of “x” is either +, or −, or 0; that is, in the expression sgn(x), “x” may have a positive value or a negative value or a zero value. Equation (2) provides the required condition so that the 3 wavelengths λ_{B}, λ_{G}, λ_{R} in the blue, green, and red channels, respectively, can provide substantially the same value for each diffraction order m when diffracted by a grating defined by Equation (1). That means that light of the 3 wavelengths upon being diffracted by a grating defined by Equation (1) can overlap on the sample.

In an embodiment, as shown in FIG. 2, broad band light of 3 different wavelengths (λ_{BX}, λ_{GX}, λ_{RX}) in which X is an integer from 1 to N representing the wavelengths in the blue (B), green (G), and red (R) wavelength bands are all coupled to or otherwise provided to illumination fiber 130, then focused by the focusing element 152 onto spacer 154 to be incident on the grating 158 at substantially the same angle θᵢ. Since grating 158 is designed to simultaneously satisfy the group of Equations (2), the 3 different wavelengths (λ_{BX}, λ_{GX}, λ_{RX}) are all diffracted at substantially the same angle θ_{d}.

Table 1 shows exemplary diffraction orders and wavelength ranges of red (R), green (G), and blue (B) diffracted light for various experimental examples where the 3 different wavelengths (λ_{BX}, λ_{GX}, λ_{RX}) were all diffracted at substantially the same angle θ_{d} by satisfying Equation (2).

TABLE 1

| | Example 1 | Example 2 | Example 3 |
|---|---|---|---|
| (m_{B}, m_{G}, m_{R}) | (−5, −4, −3) | (−6, −5, −4) | (−7, −6, −5) |
| (λ_{B}_{₁}- λ_{BN}) | (408 nm- 468 nm) | (417 nm - 479 nm) | (437 nm - 489 nm) |
| (λ_{G}_{₁}- λ_{GN}) | (510 nm - 585 nm) | (500 nm - 575 nm) | (515 nm - 570 nm) |
| (λ_{R}_{₁}- λ_{RN}) | (680 nm - 780 nm) | (625 nm - 719 nm) | (612 nm - 684 nm) |

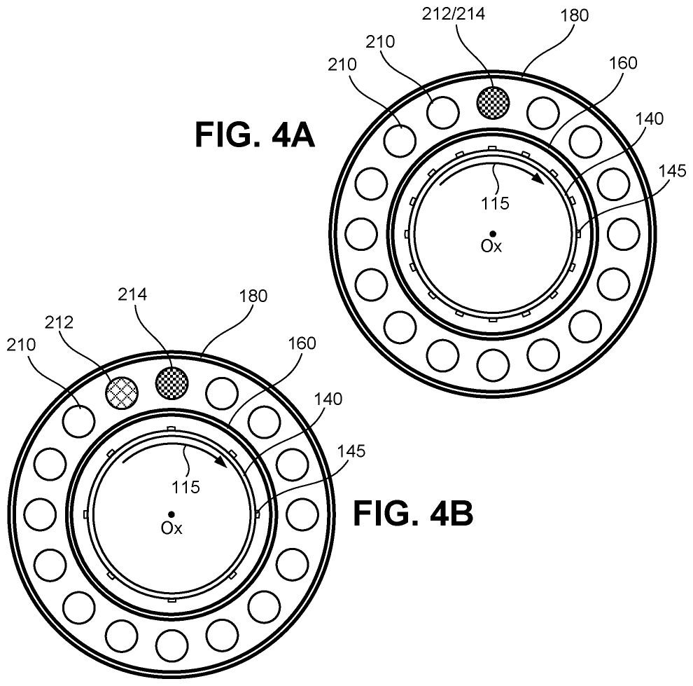

After illumination of the spectrally diffracted light 202 (e.g., red, green, and blue light) on the sample 200 (e.g., a tissue or in-vivo sample), light is reflected, scattered, photoluminescence emitted by the sample 200. This light is collected by one or plural detection fibers 210 (shown in FIG. 3). In some embodiments, there are 4, 5, 6, 7, 8, 10, or more detection fibers 210. Preferably, the detection fiber(s) 210 has a high numerical aperture (NA). The NA of fiber 210 may be more than 0.2, and more preferably the NA may be 0.3, 0.4, 0.5, 0.6, 0.7, or 0.8, or any value therebetween.

A plurality of detection fibers 210 used to collect the light reflected/emitted from the sample may be attached on or near/adjacent the outer surface of the inner sheath 160. The detection fiber 210 may be a single-mode fiber, multi-mode fiber or double clad fiber.

The probe 120 can have an outer sheath 180 outside of the inner sheath 160, so that the outer sheath 180 encloses the detection fiber 210. The probe 120 can have a window cover 162 arranged over (at the distal end of) illumination optics 150. The window cover 162 can cover also detection fiber 210. The window cover 162 is preferably made of transparent material which can be glass or plastic having high transparency and low reflectivity. The window cover 162 is preferably substantially transparent for the illumination light 202. For example, transmittance of the window cover 162 is preferably at least 50%, and more preferably it is equal to or higher than 90%.

As shown in FIG. 1, the collected light is delivered to the spectrometer 220 via the detection fiber 210. The spectrometer 220 obtains one-dimensional (1D) spectral data for the 3 wavelength bands of light (e.g., blue, green, and red light). This 1D spectral data corresponds to information from the three illumination lines (RGB) on sample 200.

The illumination fiber 130, the drivecable 140, and the probe distal optics 150 are rotated by a motor of the rotary junction 114 as indicated by the arrow 115 such that illumination light lines scan the sample in a circular manner. In this manner, the spectrometer 220 can obtain two-dimensional (2D) data (in coordinates of wavelength and time).

After the spectrometer 220 and one or more detectors detect the light collected by fiber 210, an image processor 230 generates three 2D images, one for red, green, and blue, from the data. In other embodiments, two, four or more 2D images can be formed using a probe with appropriate overlapping orders of dispersed light.

As noted above, the probe 120 is rotated by a motor of the rotary junction 114 to scan the sample in a circular manner to obtain the 2D data. Alternatively, the probe 120 may be oscillated to provide similar 2D data. The motor can be, for example, a Galvano motor, stepping motor, a piezo-electric motor, or a DC motor. Regardless of the type of actuation, by rotating or oscillating the spectrally encoded lines in the direction of the arrow 115, a substantially circular region can be imaged at a plane located at a working distance from probe.

The circular region can be located approximately perpendicular to the SEE probe axis, and therefore, the exemplary SEE probe shown in FIG. 1 can conduct forward-view imaging if m and G are chosen so that light is diffracted at a diffraction angle of θ_{d}=−θᵢ. In this manner, at least the shortest wavelength light in each color bandwidth, (λ_{R1}, λ_{G1}, λ_{B1}), can be diffracted to the sample 200 in the forward-view direction. At the spectrometer 220, the wavelength and intensity of each collected color light can be separately read out and used to generate a line image of the sample.

### <Optical Encoder>

FIG. 3 is an illustration of a portion of the SEE probe 100 showing an exemplary built-in optical encoder as it might be used in the embodiment illustrated in FIG. 1. FIG. 3 shows in detail the distal end of the probe 120. In one embodiment, the drivecable 140 or the can 142 can have one or multiple patterns 145 built-in at the distal end thereof. As used herein “built-in” is given its ordinary meaning as forming an integral part of a structure or device. That is, in the present application, the one or multiple patterns 145 are arranged on the outer surface or formed within the cylindrical wall itself of either the drivecable 140 or the can 142 so as to form an integral part of the drivecable or can structure. In one embodiment, the probe 120 also includes a first waveguide 212 and a second waveguide 214. At the distal end of probe 120, the waveguides 212 and 214 are located radially concentric to the axis Ox and in between the inner sheath 160 and the outer sheath 180. The first waveguide 212 and second waveguide 214 can be attached with an adhesive (e.g., glue, resin, tape) to the distal end of the inner sheath 160, for example. In general, the drivecable 140 and/or can 142 are configured to rotate in conjunction with the illumination optics 150 of the imaging probe 120 while the light emitting and receiving components of the encoder remain relatively static (i.e., not rotating). In this manner, the plurality of patterns formed at the distal end of the rotatable drivecable and/or can and the one or more waveguides together form a built-in optical encoder.

The first waveguide 212 can be an optical fiber which delivers second illumination light 242 from the second light source 240 to the probe 120. The waveguide 212 can have second illumination optics 216 to direct at least part of the second illumination light 242 toward the pattern 145. The patterns 145 are configured so that throughput from the first waveguide 212 to the second waveguide 214 depends on rotation position of the drivecable 140.

The second waveguide 214 can be an optical fiber and can have second detection optics 218 to receive at least part of second detection light 244 reflected from the patterns 145. The second waveguide 214 delivers the second detection light 244 to a second detector 250 (shown in FIG. 1). The second detector 250 is a photo detector which can be a line sensor shared with the spectrometer 220.

The second detector 250 detects the second detection light 244. Rotation information of the distal tip of drivecable 140 can be obtained based on the intensity variation of the detected second detection light 244 due to the rotation of patterns 145. Specifically, as the drivecable 140 rotates, the patterns 145 reflect the second illumination light 242 as the second detection light 244 at predetermined times according to the speed of rotation and the arrangement of patterns 145 with respect to the optics 216 and 218.

When 2D data (in coordinates of wavelength and time) obtained by the spectrometer 220 is turned into 2D images by the image processor 230, the rotation information of the distal tip of the drivecable 140 is used to compensate distortion of non-uniformity of rotation speed of the drivecable 140.

In one embodiment, each of waveguides 212 and 214 can be optical fibers. One or both of the fibers can be a single mode fiber so that spatial resolution of rotation information can be improved. For convenience and ease of assembly, the waveguides 212 and 214 can be the same parameters (same diameter) as detection fibers 210. The waveguides 212 and 214 are arranged concentric to the drivecable 140 and can be located next to each other or at a predetermined distance from each other.

In one embodiment, only the second illumination optics 216 and second detection optics 218 may need to be arranged concentric to the drivecable 140 and located next to each other or at a predetermined distance from each other. Then, the optics 216 and optics 218 can be fused/connected with a single fiber. The one fiber can work as both of the waveguides 212 and 214. In this case, a circulator can be used between the fiber and the second light source 240/the second detector 250 to divide the second illumination light 242 and the second detection light 244.

The second illumination optics 216 and the second detection optics 218 can be the same optics. That is, the second illumination optics 216 and the second detection optics 218 can be one set of integrated optical elements or a single optical element. The second illumination optics 216 and/or the second detection optics 218 can be a prism, a lens, a mirror, or combination of those. The second illumination optics 216 and/or the second detection optics 218 can be a common path interferometer. In one embodiment, to implement the second illumination optics 216 and/or the second detection optics 218, a focusing lens combined with a mirror or prism can be added to the distal end of a fiber 210 which functions as both of the waveguides 212 and 214. Methods well known in the art including bonding, welding, or soft-lithography (e.g., causing a pre-polymer adhesive composition to polymerize so as to form the diffractive or reflective optics on the distal end of the fiber) can be used. For example, a miniature GRIN or ball lens can be attached to the distal end of a fiber 210, and a transparent spacer can be attached to the lens, and then the spacer can be further cut or polished at an angle, and further coated with a reflective coating. In one embodiment, the second illumination optics 216 and the second detection optics 218 can be formed by one or more ball lenses having a polished surface. In this case, the polished surface serves to direct the light along a desired path, and the ball or spherical surface of the lens serves to focus and/or collimate the light.

In one embodiment, the patterns 145 can have one unique mark and other marks different from the unique mark to enable determination of absolute rotation angle. For example, the patterns 145 contain one pattern and the other pattern where the one pattern is distinguishable from the other patterns. The pattern can be periodic or non-periodic. The one pattern can be used to determine orientation of the drivecable 140 and be used to set orientation of reconstructed image in image reconstruction process. The one pattern can be used to set a zero or home position of the drivecable 140 and can be used to set an origin or home position of a reconstructed image in the image reconstruction process.

In one embodiment, the patterns 145 may have a periodic or non-periodic arrangement but not constant reflectivity in the rotation direction when the drivecable 140 rotates. For example, a specific pattern (e.g., the zero or home pattern) can have higher reflectivity that other patterns. In one embodiment, the patterns 145 may have a periodic or non-periodic arrangement but not constant directivity of reflection when the drivecable 140 rotates. For example, a specific pattern (e.g., the zero or home pattern) can have a directivity normal (perpendicular) to the second detection optics 218, while the other patterns may have an angular directivity with respect to detection optics 218.

In one embodiment, the patterns 145 may include a fluorescent material to generate periodic or non-periodic but not constant fluorescence intensity by the second illumination light 242 when the drivecable 140 rotates. For example, a specific pattern (e.g., the zero or home pattern) can have a higher concentration of fluorescent material than that the other patterns. In one embodiment, the patterns 145 are point reflectors arranged at predetermined angular spacing. In one embodiment, the patterns 145 are grooves on the drivecable 140 and/or grooves on the can 142.

In one embodiment, the patterns 145 are stripe patterns. Stripe patterns could be produced by making the encoder pattern on a planar substrate and then wrapping the substrate around the drivecable 140 or can 142 at or near the distal end of the imaging probe 120. Alternatively, stripe patterns can be formed directly on the outer surface of can 142 and/or drivecable 140 by directly machining or printing such patterns on the outer surface thereof.

In one embodiment, a combination of at least two different patterns may used. For example, a specific pattern (e.g., a point reflector) can provide a higher intensity of reflected light, which other patterns (e.g., stripes of less reflective material or grooves) can provide reflected light with decreasingly less reflectivity. In this embodiment, the specific pattern can used as the zero, home or absolute angle marker, and the other patterns can be used to determine angular, direction, and/or speed of rotation.

In one embodiment, a portion of the drivecable 140 and/or can 142 where patterns 145 exist has polygonal shape. For example, the distal end of the drivecable or the can 142 can have the shape of a polygonal mirror with a predetermined number of sides (facets) so that when the drivecable rotates each facet of the polygonal mirror reflects light to generate periodic or non-periodic but not constant intensity.

In one embodiment, it is preferable that wavelength of the second illumination light 242 to irradiate the patterns 145 be different from wavelengths of the dispersed illumination light 202 to irradiate the sample. This is considered advantageous because using different wavelengths prevents cross-talk between light used to measure NURD and light used to irradiate the sample. In addition, this prevents “direct coupling”; that is, the first 1st illumination light upon being scattered by the sample goes through the detection path to spectrometer for imaging directly.

In one embodiment, the inner sheath 160 may have a hole so that the second illumination light 242 and the second detection light 244 can go through the sheath 160. In one embodiment, the inner sheath 160 is transparent for the second illumination light 242 and the second detection light 244. In one embodiment, the inner sheath 160 is not transparent for the broadband light 116, but transparent for the light 242 and the light 244, so that broadband light 116 and the collected light 118 cannot go into the waveguide 214.

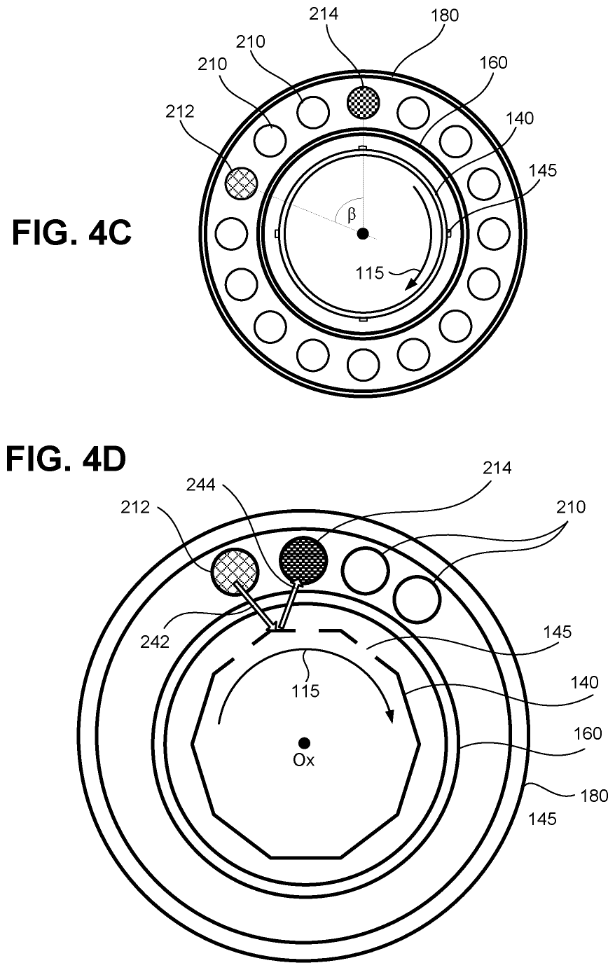

FIGS. 4A, 4B, 4C and 4D show axial views of a portion of the SEE probe too illustrating exemplary arrangements for a built-in optical encoder as it might be used in the embodiment illustrated in FIG. 1. Each of FIGS. 4A, 4B, 4C and 4D are axial or front views of the distal end of probe 120, as observed in the direction of the axis Ox from the distal end towards the proximal end of the probe. FIG. 4A shows an exemplary arrangement where the first waveguide 212 and second waveguide 214 are implemented by a single fiber (one fiber). FIG. 4B shows an exemplary arrangement where the first waveguide 212 and second waveguide 214 are implemented by two separate fibers which are arranged adjacent to each other. FIG. 4C shows an exemplary arrangement where the first waveguide 212 and second waveguide 214 are implemented by two separate fibers which are arranged concentric to the axis of the probe at a predetermined distance from each other. The predetermined distance can be determined as a function of an angle (β) to optimize the optical path for delivery and collection of the second illumination light 242 and the second detection light 244, respectively. For example, the angle (β) can be determined experimentally such that a second fiber collects the light reflected from one specific pattern 145 with the highest intensity. In addition, as illustrated in FIGS. 4A-4C, the number of patterns 145 provided on the outer surface of drivecable 140 and/or can 142 can vary according to the arrangement of the first waveguide 212 and second waveguide 214, or it can vary according to the desired level of accuracy (or resolution) in NURD measurement. FIG. 4D shows an exemplary embodiment where the distal end of the drivecable 140 or the can 142 is formed in the shape of a reflecting polygon (polygonal mirror) with a predetermined number of sides (facets). Each facet includes at least one of patterns 145. In this embodiment, the patterns 145 are hollow indentations (groves, slits or slotted openings) formed integrally on the wall of the polygon shaped mirror. In this manner, when the drivecable 140 rotates in direction 115, each facet of the polygonal mirror is illuminated by second illumination light 242 from second illuminating optics 212, and the mirror reflects light as second detection light 244 towards collecting optics of the second waveguide 214. In this embodiment, during rotation of drivecable 140, the patterns 145 inhibit reflection of light, and therefore periodic or non-periodic bursts of light can be collected and processed.

### <Encoder Operation>

During operation of the SEE imaging probe 100, the second illumination source 240 delivers second illumination light 242 to the patterns 145 via the first waveguide 212 and the second illumination optics 216 to make at least part of the second illumination light 242 go to the pattern 145. The second detection optics 218 receive at least part of the second detection light 244 reflected from the patterns 145, and the second waveguide 214 delivers the second detection light 244 to a second detector 250.

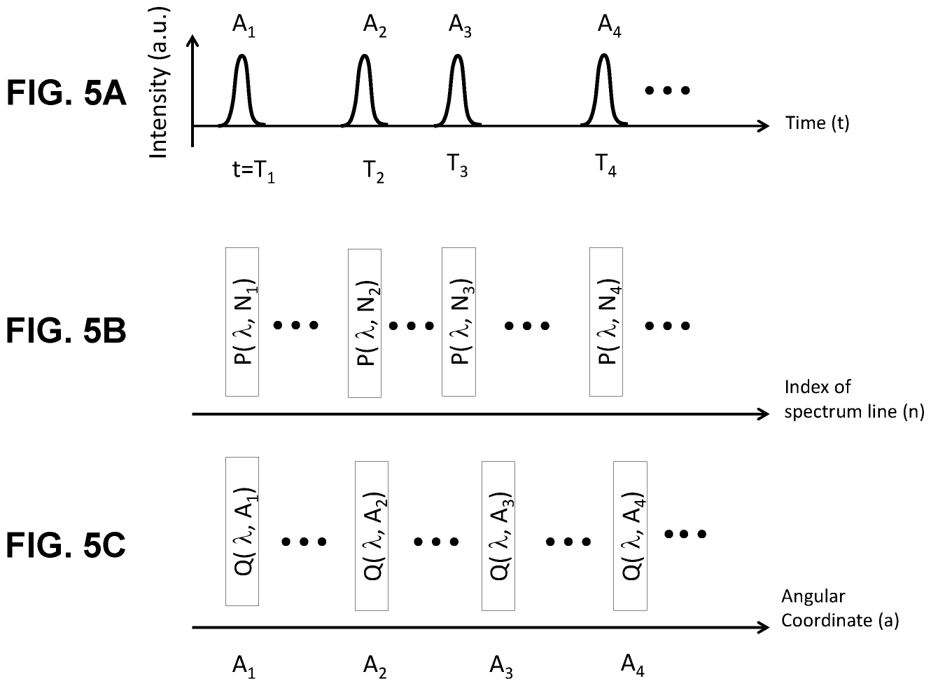

FIG. 5A shows the intensity modulation detected at the second detector 250 as a function of time. Here the amplitude a=Aᵢ (i=1, 2, 3, . . . ) of a intensity modulation peak corresponding to a rotation angle of the pattern 145 is detected at time t=Tᵢ (i=1, 2, 3, . . . ). Even though the rotation angle of the pattern 145 may be set at regular intervals, if there is non-uniform rotation on the probe (e.g., due to friction), the time Tᵢ at which the amplitude Aᵢ is detected is not at regular intervals. That is, as shown in FIG. 5A, although the amplitude λₗ of bursts of light collected during probe rotation may be uniform, the timing at which the light is collected may not be uniform. Therefore, as shown in FIG. 5A, the amplitude of the intensity modulation detected at the second detector 250 can be collected at non-uniform time intervals.

FIG. 5B shows the 2D data obtained at spectrometer 220 as a function of wavelength (spectrum). The 2D data obtained at the spectrometer 220 is P(λ, n) where λ is wavelength and n is an index of each spectrum line. Here we define indices of spectrum line corresponding time Tᵢ (i=1, 2, 3, . . . ) as n=Nᵢ (i=1, 2, 3, . . . ). The goal of this step of imaging process is to convert the 2D data from P(, n), where each spectrum line is obtained in equal interval in time-domain, to Q(λ, a), where each spectrum line is obtained in equal intervals in angle-domain in the polar coordinate system.

FIG. 5C shows the converted 2D data as function of angular position (in angular coordinates) as the drivecable 140 rotates. As it can be appreciated from FIG. 5C, the converted 2D data Q(λ, Aᵢ) is now displayed at substantially equal (regular) intervals.

The polar coordinate system is defined based on the view angle and distance from probe axis. One method to obtain Q(λ, a) is to apply linear compensation to P(λ, n). For example, if Q(λ, a) is defined such that it has AA spectrum lines between Q(λ, Aᵢ) and Q(λ, Aᵢ₊₁) for any i, Q(λ, a) between Q(λ, Aᵢ) and Q(λ, λᵢ₊₁), ΔA spectrum lines, are calculated by applying linear compensation to the spectrum lines of P(λ, n) between P(λ,Nᵢ) and P(λ,Nᵢ₊₁). It should be noted here that Nᵢ₊₁−Nᵢ depends on i and is not constant when rotation is not uniform. However, by applying linear compensation to P(λ, n), the converted 2D data Q(λ, Aᵢ) is obtained at substantially equal (regular) intervals.

### <Imaging Operation and Image Processing>

The SEE probe system 100 shown in FIG. 1 is generally used for imaging bodily limens under the operation of a micro-computer or image processor 230. The computer of image processor 230 includes one or more microprocessors configured to control and operate the various parts of SEE probe system 100, and executes computer-executable instructions (program code) to reconstruct images based on signals obtained from probe 120. To acquire an image with the SEE probe system 100, according to an exemplary embodiment of the present disclosure, the image processor 230 controls the first light source 110 to output light of a broadband spectrum (broadband light 116), which is directly guided or otherwise transported by fiber 112 to the sample 200 via the FORJ 114 and the probe 120. The light scattered back from an object or sample 200 (e.g., tissue) can be collected by detection fibers 210 and guided to the spectrometer 220. By mechanically scanning the probe 120 in a rotating direction 115 using a mechanical scanning unit contained within the FORJ 114, it is possible to obtain a two-dimensional image of the object. The mechanical scan can be performed by, e.g., Galvo scanner or motor to rotate the drivecable 140 together with the illumination optics contained therein.

In some exemplary embodiments, instead of guiding the broadband light from light source 110 into the illumination fiber 112, the light can first be dispersed to predetermined wavelength(s) λ₁, λ₂, . . . , λ_{N}. For example, the light with the wavelength λi (1≤i≤N) can be input into the illumination fiber 112 in a multiplexed manner. The input light is provided through the junction (FORJ 114), illumination fiber 130, distal optics 150 to the sample 200; and collected via the fibers 210 and guided to the detector/spectrometer 220. Optionally, in the case of imaging with light of individual wavelengths λi, the detector/spectrometer 220 can be or include a simple light intensity detector such as photodetector because the input light has a wavelength of λi. Then, by changing the wavelength i from 1 to N, it is possible to obtain the one-dimensional line image at different wavelengths, by using a simple intensity photodetector or a line sensor. By mechanically scanning the line, it is possible to acquire the two-dimensional image of the object.

One function of the fiber junction (FORJ 114) is to make the probe 120, including the illumination fiber 130 and distal optics 150, detachable. With this exemplary function, the probe 120 can be disposable and thus a sterile probe for human “in vivo” use can be provided every time an imaging operation is performed. In some embodiments, the probe 120 is a disposable probe. In these embodiments, the illumination fiber 130, the detection fiber 210, and the waveguides 212/214 may be detachable. With this exemplary function, the probe 120 may be disposable in order to ensure that a sanitary probe is being inserted into the subject which may be a human body.

The SEE probe system 100 as described and shown herein can diffract the broadband light along the axis of the probe 120, and facilitate forward viewing. The exemplary probe may be held stationary or it may be rotated, where the rotation of the probe is particularly useful for acquiring a two-dimensional front-view image as well as a color image. Since the detection fibers 210 can be attached to the outer surface of the non-rotatable inner sheath 160, continuous rotation of the probe 120 is possible without the illumination fiber 130 and distal optics 150 becoming with other instruments. However, in in some exemplary applications, it may be necessary that the probe 120 is to be rotated in an oscillating manner, e.g., +/− approximately 360 degrees back and forth. In other exemplary situations, the probe 120 can be rotated +/− approximately 180 degrees back and forth. In further exemplary embodiments, other degrees of oscillating rotation can be used, such as, e.g., 90 degrees or 270 degrees of back and forth rotation.

Whether the probe 120 is rotated continuously or in an oscillating manner, the possibility of non-uniform rotational distortion (NURD) cannot be avoided. Therefore, it is important to implement accurate NURD measurement. To that end, at the same time as the imaging operation takes plane, the image processor 230 controls the second light source 240 to emit second illumination light 242 which is directly guided or otherwise transported by the first waveguide 212 to the patterns 145. Upon reflection from the patterns 145, the reflected light (second detection light 244) is guided by the second waveguide 214 to the second detector 250. The output of the second detector 250 will then be used by the processor 230 to estimate or measure the rotational motion of the probe 120 and any distortions thereof. In this manner, the second light source 240 and second detector 250 in cooperation with the waveguides 212/214, the optics 216/218, and the patterns 145 form an optical encoder circuit.

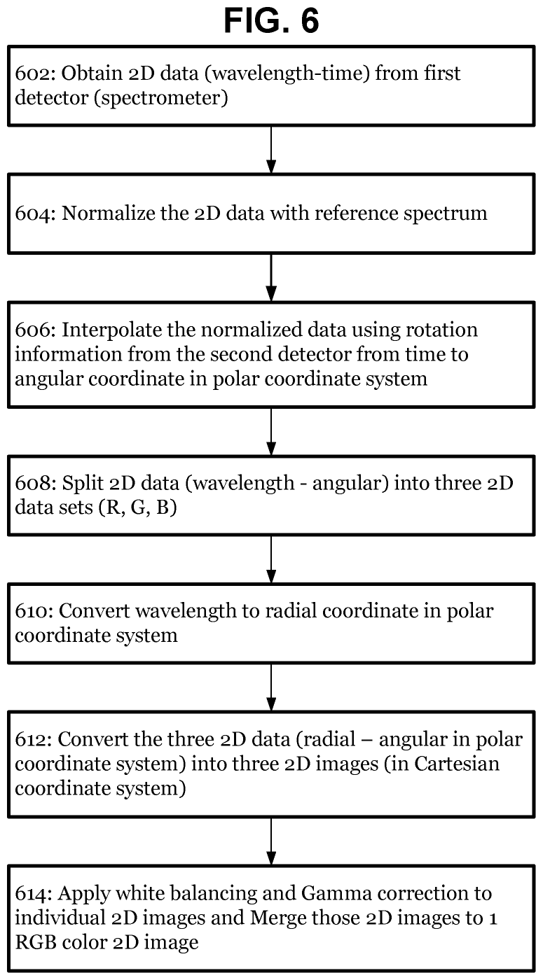

FIG. 6 shows an exemplary block diagram of image processing and NURD measurement executed by the image processor 230. The process of FIG. 6 assumes the status of SEE probe system 100 is under normal operation, and a command to perform an imaging operation and measure non-uniform rational distortion has been issued/input by an operator. At step 602, 2D data (image data) as a function of wavelength and time (wavelength-time) is obtained at spectrometer 220. At step 604, the image processor 230 normalizes the 2D data by a reference spectrum. This normalization is done to compensate throughput difference from wavelength to wavelength, such as spectrum of the broadband light source 110, coupling efficiency spectrum of the rotary junction 114, diffraction efficiency of grating 158, detection efficiency of detection fiber 118, throughput of the spectrometer 220, and so on. The reference spectrum can be obtained by averaging 2D data in time (as function of time) obtained by using the same imaging SEE probe system 100 and a white target for imaging, for example.

Then the normalized 2D data (in wavelength-time coordinates) is converted to 2D data (wavelength—angular coordinate in polar coordinate system) using rotation information from the second detector 250. As explained above, the drivecable 140 or the can 142 at the tip of the drivecable 140 has the multiple patterns 145 which have periodic reflectance in the rotation direction. However, even if the rotation angle of the pattern 145 is set at regular intervals, if there is non-uniform rotation, the image data is collected at times Tᵢ of non-uniform intervals. Therefore, at step 606, the image processor 230 interpolates the normalized data (normalized image data) using rotation information data (rotation data) from the second detector 250.

Next, at step 608, the image processor 230 splits the image data in polar coordinates to extract three (3) 2D data sets, one for each channel (Red, Green, and Blue channels) from the 2D data calculated in step 606 based on wavelength of the three channels. In the case of single color image, the image data in polar coordinates may be split in different 2D data sets according to diffraction orders of the single color.

At step 610, the image processor 230 converts each 2D data set in wavelength domain into 2D data in radial coordinate domain in polar coordinate system. To do this conversion, one method is to know the relation between wavelength and radial coordinate in polar coordinate system and apply linear compensation so that the converted image data is set at regular intervals in the radial coordinate domain. To know the relation between wavelength and radial coordinates, probe design parameters like grating groove density and incident angle can be used. Conversion from wavelength (λ) domain to radial (θ_{d}) domain can be done based on Equation (1) by the following steps. First, create θ_{d} array at regular intervals. Second, convert each θ_{d} to wavelength using Equation (1) and make a wavelength array (at irregular intervals). Third, calculate linear compensation coefficients between the wavelength array and wavelengths for obtained data (wavelengths corresponding to spectrometer pixels). Fourth, convert obtained data to linear compensated data using the coefficients of linear compensation.

At step 612, the processor 230 converts the three 2D data in polar coordinate system to three 2D images in Cartesian coordinate system. At step 614, the processor 230 can apply white balancing and gamma correction to each of the individual 2D image data sets. After applying the necessary corrections, the processor 230 merges the individually corrected 2D image data sets into one RGB color 2D image.

Certain aspects of the present disclosure be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like. An I/O interface can be used to provide communication interfaces to input and output devices, which may include a keyboard, a display, a mouse, a touch screen, touchless interface (e.g., a gesture recognition device) a printing device, a light pen, an optical storage device, a scanner, a microphone, a camera, a drive, communication cable and a network (either wired or wireless). Also, the function of one or more detectors may be realized by computer executable instructions (e.g., one or more programs) recorded on a Storage/RAM.

FIG. 1B shows a schematic block diagram of a micro-computer or image processor applicable to the various aspects of the SEE probe system 100 described herein. As shown in FIG. 1B, the computer or image processor 230 includes central processing unit (CPU) 2301, a storage memory (RAM) 2302, a user input/output (I/O) interface 2303, and a system interface 2304. The computer or image processor 230 can programmed to issue a command that can be transmitted to the various elements of the SEE probe system 100 upon receiving a user input via the user interface 2303. A touch panel screen can be included as part of the user interface 2303, but key board, mouse, joy-stick, ball controller, and foot pedal can also be included too. The user can cause a command to be initiated to observe inside a lumen of a human body through the exemplary front-view SEE probe using the user interface. For example, when the user inputs a command via the user interface 2303, the command is transmitted to the central processing unit CPU 2301 for execution thereby causing the CPU 2301 to issue a command via the system interface 2304 to one or more of the first light source 110, the second light source 240, detector/spectrometer 220, the second detector 250, or FORJ 114.

The CPU 2301 is comprised of one or more processors (microprocessors) configured to read and perform computer-executable instructions stored in the storage memory 2302. The computer-executable instructions may include program code for the performance of the novel processes, methods and/or calculations disclosed herein.

The computer or image processor 230 can be programmed to apply exemplary image processing such as noise reduction, coordinate distortion correction, contrast enhancement and so on. After or even during the image processing is performed, the data can be transmitted from the image processor to a display (not shown). A liquid crystal display (LCD) can be the display. The display can display, for example, the individual images obtained from a single color or a composite color image according to the present disclosure. The display can also display information other than the image, such an amount of NURD, the date of observation, what part of the human body is observed, the patient's name, operator's name and so on.

The CPU 2301 is configured to read and perform computer-executable instructions stored in the Storage/RAM 2302. The computer-executable instructions may include those for the performance of the methods, measurements, and/or calculations described herein. For example, CPU 2301 may calculate the angular movement or speed of rotation of the SEE probe, and can use that information (rotation speed or angular movement) to calculate an amount of NURD, and it can also control the FORJ 114 view of the amount of NURD. In this manner, computer or processor 230 can obtain a new set of images where their angular positions are corrected and the amount of NURD is reduced or eliminated. Storage memory 2302 includes one or more computer readable and/or writable media, which may include, for example, a magnetic disc (e.g., a hard disk), an optical disc (e.g., a DVD, a Blu-ray), a magneto-optical disk, semiconductor memory (e.g., a non-volatile memory card, flash memory, a solid state drive, SRAM, DRAM), an EPROM, an EEPROM, etc. Storage/RAM 402 may store computer-readable data and/or computer-executable instructions. The components of the processor 230 may communicate via a data bus 2305.

The system interface 2304 provides communication interfaces to input and output devices, which may include a keyboard, a display, a mouse, a printing device, a touch screen, a light pen, an optical storage device, a scanner, a microphone, a camera, a drive, communication cable and a network (either wired or wireless). The system interface 2304 also provides communication interfaces to input and output devices.

The second detector 250 may include, for example a photomultiplier tube (PMT), a photodiode, an avalanche photodiode detector (APD), a charge-coupled device (CCD), multi-pixel photon counters (MPPC), or other like element. Also, the function of detector 250 may be realized by the processor 230 operating on computer executable instructions (e.g., one or more programs) recorded on storage memory 2302.

In an exemplary operation of the SEE probe system 100, the user/operator can place the exemplary SEE probe 120 into a sheath, and then can insert such arrangement/configuration into a predetermined position of a human body. The sheath alone may be inserted into the human body in advance, and it is possible to insert the SEE probe into the sheath after sheath insertion. The exemplary probe can be used to observe inside human body and works as endoscope such as arthroscopy, bronchoscope, sinuscope, vascular endoscope, and so on.

### Definitions

In referring to the description, specific details are set forth in order to provide a thorough understanding of the examples disclosed. In other instances, well-known methods, procedures, components and circuits have not been described in detail as not to unnecessarily lengthen the present disclosure.

It should be understood that if an element or part is referred herein as being “on”, “against”, “connected to”, or “coupled to” another element or part, then it can be directly on, against, connected or coupled to the other element or part, or intervening elements or parts may be present. In contrast, if an element is referred to as being “directly on”, “directly connected to”, or “directly coupled to” another element or part, then there are no intervening elements or parts present. When used, term “and/or”, includes any and all combinations of one or more of the associated listed items, if so provided.

Spatially relative terms, such as “under” “beneath”, “below”, “lower”, “above”, “upper”, “proximal”, “distal”, and the like, may be used herein for ease of description to describe one element or feature's relationship to another element(s) or feature(s) as illustrated in the various figures. It should be understood, however, that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, a relative spatial term such as “below” can encompass both an orientation of above and below. The device may be otherwise oriented (rotated 90 degrees or at other orientations) and the spatially relative descriptors used herein are to be interpreted accordingly. Similarly, the relative spatial terms “proximal” and “distal” may also be interchangeable, where applicable.

The term “about,” as used herein means, for example, within 10%, within 5%, or less. In some embodiments, the term “about” may mean within measurement error.

The terms first, second, third, etc. may be used herein to describe various elements, components, regions, parts and/or sections. It should be understood that these elements, components, regions, parts and/or sections should not be limited by these terms. These terms have been used only to distinguish one element, component, region, part, or section from another region, part, or section. Thus, a first element, component, region, part, or section discussed below could be termed a second element, component, region, part, or section without departing from the teachings herein.

The terminology used herein is for the purpose of describing particular embodiments only and is not intended to be limiting. As used herein, the singular forms “a”, “an”, and “the”, are intended to include the plural forms as well, unless the context clearly indicates otherwise. It should be further understood that the terms “includes” and/or “including”, when used in the present specification, specify the presence of stated features, integers, steps, operations, elements, and/or components, but do not preclude the presence or addition of one or more other features, integers, steps, operations, elements, components, and/or groups thereof not explicitly stated.

In describing example embodiments illustrated in the drawings, specific terminology is employed for the sake of clarity. However, the disclosure of this patent specification is not intended to be limited to the specific terminology so selected and it is to be understood that each specific element includes all technical equivalents that operate in a similar manner.

While the present disclosure has been described with reference to exemplary embodiments, it is to be understood that the present disclosure is not limited to the disclosed exemplary embodiments. The scope of the following claims is to be accorded the broadest interpretation so as to encompass all such modifications and equivalent structures and functions.

What is claimed is:

1. An endoscopic optical probe, comprising:
a rotatable core for guiding first illumination light from a first light source towards an imaging target, the rotatable core extending from a proximal end to a distal end along an axis and having a plurality of patterns built-in at the distal end thereof;
a non-rotatable sheath arranged concentrically around the rotatable core and also extending from the proximal end to the distal end; and
one or more optical waveguides disposed on the outer surface of the non-rotatable sheath at the distal end thereof,
wherein the one or more waveguides are configured to guide second illumination light from a second light source onto the plurality of patterns and collect light reflected from the plurality of patterns,
wherein the wavelength of the first illumination light is different from the wavelength of light of the second illumination light,
wherein the plurality of patterns are rotationally aligned with the waveguides such that the waveguides collect the reflected light as a function of a rotation position of at least first and second patterns on the rotatable core, and
wherein the rotation position is determined by intensity variation the reflected light.

2. The endoscopic optical probe according to claim 1, wherein the one or more waveguides are configured to guide the second illumination light onto the plurality of patterns and collect the reflected light from the plurality of patterns while the rotatable core rotates or oscillates around the axis.

3. The endoscopic optical probe according to claim 2, wherein the plurality of patterns formed at the distal end of the rotatable core and the one or more waveguides arranged at the distal end of the non-rotatable sheath together form a built-in optical encoder in the probe, and wherein the optical encoder is configured to encode rotational speed and/or angular displacement of the rotatable core.

4. The endoscopic optical probe according to claim 1, wherein the rotatable core includes a light guiding component, a light focusing component, and a grating component arranged in this order along the axis, and wherein the light focusing component and the grating component are configured to irradiate the imaging target with the first illumination light.

5. The endoscopic optical probe according to claim 4, further comprising a plurality of detection optical fibers arranged around the distal end of the non-rotatable sheath.

6. The endoscopic optical probe according to claim 5,
wherein some of the detection optical fibers are configured to collect first detection light scattered by the imaging target upon irradiation with the first illumination light; and
wherein one or more of the detection optical fibers are the one or more waveguides configured to guide the second illumination light from the second light source onto the plurality of patterns and collect light reflected from the plurality of patterns upon irradiation with the second illumination light.

7. The endoscopic optical probe according to claim 1,
wherein the plurality of patterns are arranged concentric with and parallel to the axis,
wherein the plurality of patterns are configured to have periodic or non-periodic non-uniform reflectivity in the rotation direction of the rotatable core, and/or are configured to have periodic or non-periodic non-uniform directivity of reflection in the rotation direction of the rotatable core, such that the plurality of patterns generate a non-uniform intensity signal when the rotatable core rotates.

8. The endoscopic optical probe according to claim 1, wherein the plurality of patterns includes different amounts of fluorescent material or different amounts of reflective material such, upon irradiation by the second illumination light, the plurality of patterns generate a periodic or non-periodic light signal having not constant intensity when the rotatable core rotates.

9. The endoscopic optical probe according to claim 1, wherein the plurality of patterns respectively includes a plurality of point reflectors, or a plurality of grooves, or a plurality of stripe patterns, or a combination of at least one of each of point reflectors, grooves, and stripe patterns.

10. The endoscopic optical probe according to claim 1, wherein the distal end of the rotatable core has a polygonal shape, and wherein the plurality of patterns are formed on the sides of the polygonal shape or are the sides of the polygonal shape.

11. The endoscopic optical probe according to claim 1,
wherein the non-rotatable sheath has a transparent section and/or has a hole at the distal end thereof so that the second illumination light and/or the reflected light can go through the transparent section or through the hole of the non-rotatable sheath.

12. The endoscopic optical probe according to claim 6,
wherein the first illumination light is broadband light, and
wherein the non-rotatable sheath is not transparent for the broadband light, so that neither the broadband light nor the scattered light can transmit through the non-rotatable sheath.

13. The endoscopic optical probe according to claim 1,
wherein the plurality of patterns includes one specific pattern and other patterns different from the one specific pattern,
wherein the one specific pattern is optically distinguishable from the other patterns; and
wherein the one specific pattern is configured to determine an angular orientation of the rotatable core or to set a zero or home position of the rotatable core.

14. The endoscopic optical probe according to claim 1,
wherein the plurality of waveguides are included in one or more optical fibers, and
wherein at least one of the one or more optical fibers is a single mode fiber.

15. The endoscopic optical probe according to claim 1,
wherein the one or more waveguides includes a pair of optical fibers arranged on the outer surface of the non-rotatable sheath at a predetermined distance from each other,
wherein the predetermined distance is determined by an angle formed between a first fiber illuminating the patterns with the second illumination light and a second fiber collecting the reflected light from the patterns, and
wherein the second fiber is arranged at a position where the reflected light is collected with the highest intensity while the rotatable core rotates.

16. An endoscopic imaging system comprising:
the endoscopic optical probe according to claim 3; and
a processor configured to calculate an amount of non-uniform rotational distortion based on the rotational speed and/or angular displacement of the rotatable core.

17. The endoscopic imaging system according to claim 16,
further comprising a first detector configured to detect intensity of light scattered by the imaging target and collected by the plurality of optical fibers arranged around the distal end of the non-rotatable sheath,
wherein the processor is further configured to generate an image of the imaging target based on detected intensity of light scattered by the imaging target.

18. The endoscopic imaging system according to claim 17,
wherein the processor is further configured to generate image data in wavelength and time coordinates, and to normalize the image data of the imaging target using a reference spectrum.

19. The endoscopic imaging system according to claim 18,
further comprising a second detector configured to detect intensity of the light reflected from the plurality of patterns,
wherein the processor is further configured to obtain rotation data based on the intensity of light reflected from the plurality of patterns, and to interpolate the normalized data from time to angular coordinates using the rotation data from the second detector.

20. The endoscopic imaging system according to claim 19,
wherein the processor is further configured to split the interpolated data into a plurality of separate data sets having wavelength and angular coordinates.

21. The endoscopic imaging system according to claim 20,
wherein the processor is further configured to convert the wavelength coordinates of each data set to radial coordinates in a polar coordinate system.

22. The endoscopic imaging system according to claim 21,
wherein the processor is further configured to convert the plurality of data sets having radial and angular coordinates into a corresponding plurality of two-dimensional images having a Cartesian coordinate system.

23. The endoscopic imaging system according to claim 22,
wherein the processor is further configured to apply white balancing and gamma correction to each of the two-dimensional images, and merge the two-dimensional images into a single image.

24. The endoscopic optical probe according to claim 1,
wherein optical probe is a forward viewing spectrally encoded endoscopy (SEE) probe,
wherein the rotatable core guides the first illumination light towards the imaging target such that at least part of the first illumination light travels substantially parallel to the axis, and
wherein the one or more waveguides are configured to guide the second illumination light and collect the light reflected from the plurality of patterns in a direction substantially perpendicular to the axis.
